(19) United States
(12) Patent Application Publication
(10) Pub. No.: US 2007/0118152 A1
(43) Pub. Date: May 24, 2007

(54) Surgical suture passer

(76) Inventor: Thomas Calvin Page, Covington, LA (US)

Correspondence Address: Thomas C. Page, 214 Cane Court, Covington, LA, 70433, US

(21) Appl. No.: 11/283,281

(22) Filed: Nov. 21, 2005

(51) Int. Cl. A61B 17/04

(52) U.S. Cl. 606/148

(57) ABSTRACT

A spring-loaded, cylindrical suture passer with hand grip and release button, capable of firmly grasping suture within a clamping inner cannula, particularly useful in facial plastic and head and neck surgery. The suture passer's design facilitates smaller entry incisions and less subcutaneous tissue dissection in order to pass sutures through soft tissues, thereby reducing post-operative swelling and recovery time.

### CROSS-REFERENCE TO RELATED APPLICATIONS

U.S. Pat. No. 5,320,633 Jun. 14, 1994 Allen, et al.
U.S. Pat. No. 5,467,786 Nov. 21, 1995 Allen, et al.
U.S. Pat. No. 4,006,747 Feb. 8, 1997 Kronenthal, et al.
U.S. Pat. No. 5,085,661 Feb. 4, 1992 Moss
U.S. Pat. No. 4,126,124 Nov. 21, 1978 Miller
U.S. Pat. No. 4,144,876 Mar. 20, 1979 DeLeo
U.S. Pat. No. 4,235,238 Nov. 25, 1980 Ogiu, et al.
U.S. Pat. No. 4,935,027 Jun. 19, 1990 Yoon

### BACKGROUND OF THE INVENTION

Many surgical procedures involve the use of sutures passed through soft tissue for tissue fixation to bone or fascia, intended for cosmetic tissue repair or suspension, or tissue suspension to regain lost lumen patency.

The surgeon repairing or suspending tissue needs to facilitate passing sutures accurately through soft tissue, thereby retrieving the suture at a separate incision site for fixation to bone or fascia. There are prior art devices used to facilitate either or both of these processes, of which the current proposed art offers unique improvements and benefits.

U.S. Pat. No. 5,320,633 (Allen, et al.) discloses a tube for passing suture for meniscus repair. The suture passer tubes are not fixed by spring activation, but the pushrod is passed within an external cylinder when needed.

Another prior art device is disclosed by Kronenthal , et al. (U.S. Pat. No. 4,006,747), describing a method for closing wounds and surgical incisions in mammalian tissue which comprises fastening an approximated wound or incision with a series of devices comprising a short filament with a head on each end, each device being surgically placed so that the filament traverses the wound or incision within the tissue while one head of the device engages the surface of the tissue on either side of the wound or incision.

Miller (U.S. Pat. No. 4,126,124) teaches a method for implanting hair into human skin comprising the steps of tying a knot into a hair with the knot defining an interstice through which skin tissue may subsequently grow thereby anchoring the hair firmly in place, loading a needle by placing the hair knot in a hollow end of the needle and extending the hair from the knot through a notch formed in a surface of the needle end, inserting the loaded needle into the skin, and extracting the needle from the skin.

Similarly, DeLeo (U.S. Pat. No. 4,144,876) discloses an apparatus and method for implanting hair or hair-like strands in areas of the skin of animate beings, comprising a unique fabricated anchorage piece and a novel process for placing the same in the skin, utilizing a hypodermic needle having a grooved barrel and tip portion.

For endoscopic applications, Ogiu et al. (U.S. Pat. No. 4,235,238) presents a coeliac tissue-suturing apparatus comprising a flexible tubular member having a passage extending through and inserted into a channel of an endoscope.

U.S. Pat. No. 5,085,661 (Moss) discloses a fastener implanting device used to implant a head portion of a “T” or “H”-shaped fastener within a body. The device has a needle portion and a grip portion. The needle portion has a fastener receiving cavity selectively covered by a movable sleeve. The sleeve's translational movement is controlled by a mechanism located in the device's grip portion. This device incorporates a straight shaft with a locking mechanism to either grasp or release suture.

There is a need for a delivery device which allows for more precise delivery of suture or retrieval of suture from one incision site to another, thus achieving a greater grasping strength than the prior arts as well as ease of suture threading in confined spaces. The device's trocar shaft must be flexible enough to allow the surgeon to torque the trocar shaft upwards of forty (40) degrees.

Similarly, DeLeo (U.S. Pat. No. 4,144,876) discloses an apparatus and method for implanting hair or hair-like strands in areas of the skin of animate beings, comprising a unique fabricated anchorage piece and a novel process for placing the same in the skin, utilizing a hypodermic needle having a grooved barrel and tip portion.

For endoscopic applications, Ogiu et al. (U.S. Pat. No. 4,235,238) presents a coeliac tissue-suturing apparatus comprising a flexible tubular member having a passage extending through and inserted into a channel of an endoscope.

U.S. Pat. No. 5,085,661 (Moss) discloses a fastener implanting device used to implant a head portion of a “T” or “H”-shaped fastener within a body. The device has a needle portion and a grip portion. The needle portion has a fastener receiving cavity selectively covered by a movable sleeve. The sleeve's translational movement is controlled by a mechanism located in the device's grip portion. This device incorporates a straight shaft with a locking mechanism to either grasp or release suture.

There is a need for a delivery device which allows for more precise delivery of suture or retrieval of suture from one incision site to another, thus achieving a greater grasping strength than the prior arts as well as ease of suture threading in confined spaces. The device's trocar shaft must be flexible enough to allow the surgeon to torque the trocar shaft upwards of forty (40) degrees.

### DESCRIPTION OF DRAWINGS

FIG. 1

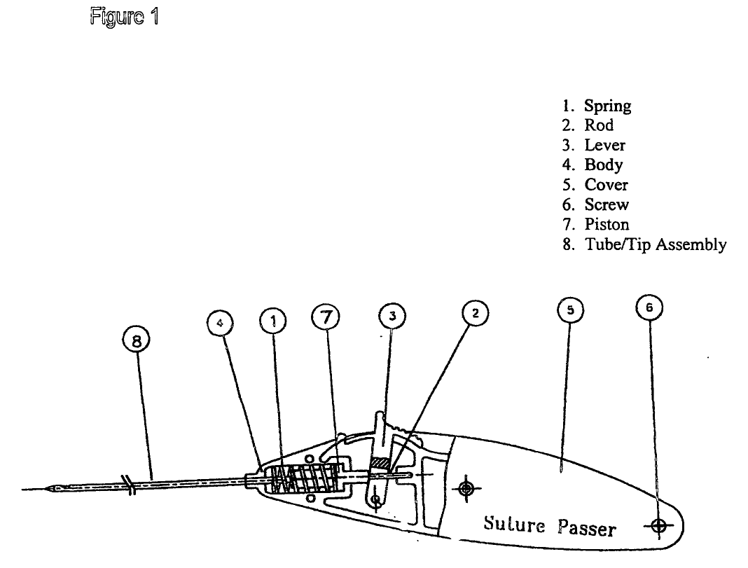

Depicting an spring activation mechanism and cylinder, internalized within the hand piece,

A pushrod within a tube cylindrical or square shaft,

A grasping and release trigger, incorporated on the top of the hand piece,

A unique tube tip assembly, for dissecting through soft tissue, incorporating an internal proximal platform jaw to capture and affix a suture tail.

FIG. 2

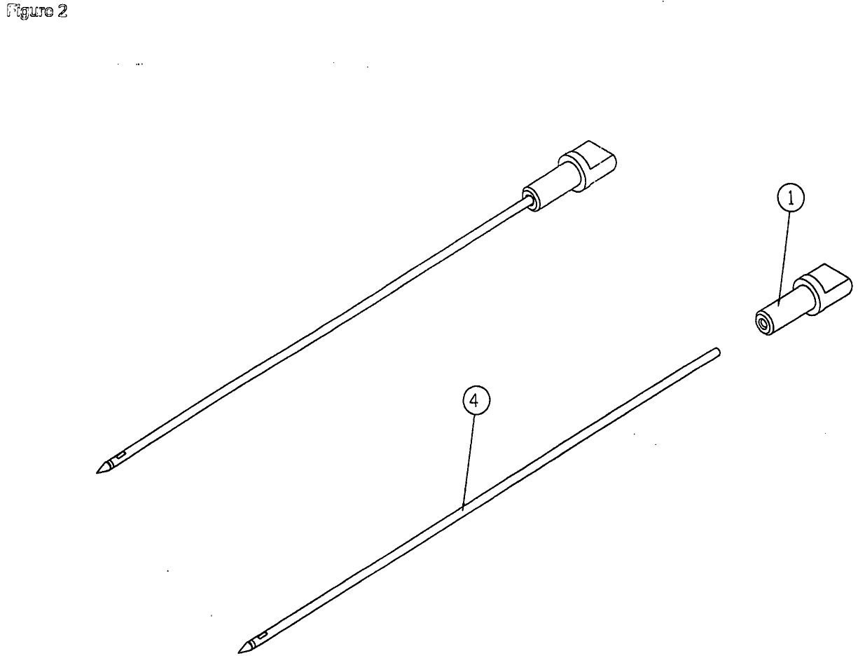

Depicting a pushrod within a tube shaft assembly,

A unique tube tip assembly, for dissecting through soft tissue, incorporating an internal proximal platform jaw to capture and affix a suture tail,

A port window in the outer shaft, for placing a suture tail between the jaws of the proximal tube tip assembly and the distal tip of the inner pushrod.

What is claimed is:

1. A suture passer device, comprising:
A handle,
Designed to fit ergonomically within the grasp of the surgeon's hand, or held between the surgeons grasping fingers,
A device with a low profile activation button on the handle, mounted on top of the handle, to be activated by the surgeon's thumb or index finger.
A device incorporating a spring loaded external tube with an internal pushrod,
A device wherein the outer shaft incorporates a piercing tube tip assembly with a suture grasping port posterior to the piercing tip.

2. The device of claim 1, wherein said grasping port is located within the proximal end of the piercing tube tip assembly.

3. The device of claim 1, wherein the inner pushrod tube is activated by an internalized spring mechanism designed to grasp a suture tail within a cannulae.

4. The device of claim 1, wherein the outer shaft may be either straight or curved, depending on the surgical and anatomic application.

5. The device of claim 1, wherein the piercing tube tip assembly may be conical, marlin spiked, round or blunt.

6. The device of claim 1, wherein the outer shaft can be manufactured in different lengths, depending upon the surgical or anatomic application intended.

7. The device of claim 4, wherein the shaft is flexible enough for the surgeon to torque the shaft upwards of forty (40) degrees, while still maintaining the integrity of the shaft and its ability to grasp suture.

8. The device of claim 2, wherein the grasping port incorporates a unique platform which allows suture to be held with greater strength than the devices of prior art.
